(19) United States
(12) Patent Application Publication
(10) Pub. No.: US 2010/0298202 A1
(43) Pub. Date: Nov. 25, 2010

(54) BRI POLYPEPTIDES AND REDUCING AB AGGREGATION

(75) Inventors: Karen R. Jansen-West, Jacksonville Beach, FL (US); Todd E. Golde, Ponte Vedra Beach, FL (US); Jungsu Kim, Jacksonville, FL (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 12/667,976

(22) PCT Filed: Jul. 2, 2008
(86) PCT No.: PCT/US2008/069084

§ 371 Date: Aug. 6, 2010

Related U.S. Application Data
(60) Provisional application No. 60/948,407, filed on Jul. 6, 2007.

(51) Int. Cl. A61K 38/00; A61P 25/28; A61K 31/7088

(52) U.S. Cl. 514/1.1; 514/44 R

(57) ABSTRACT

This document relates to methods and materials for reducing Aβ aggregation. For example, methods and materials related to the use of BRI polypeptides (e.g., BRI2 polypeptides) and fragments of BRI polypeptides (e.g., a BRI23 polypeptide) to reduce Aβ aggregation in mammals are provided.

### CROSS-REFERENCE TO RELATED APPLICATIONS

This application claims the benefit of U.S. Provisional Application Ser. No. 60/948,407, filed Jul. 6, 2007. The disclosure of the prior applications is considered part of (and is incorporated by reference in) the disclosure of this application.

### BACKGROUND

1. Technical Field

This document relates to methods and materials for reducing Aβ aggregation. For example, this document provides methods and materials related to the use of BRI polypeptides (e.g., BRI2 polypeptides) and fragments of BRI polypeptides (e.g., BRI2 polypeptides) to reduce Aβ aggregation in mammals.

2. Background Information

Familial British and Danish dementias (FBD and FDD, respectively) are neurodegenerative dementias pathologically characterized by parenchymal preamyloid and amyloid deposits, cerebral amyloid angiopathy (CAA), neuronal loss and neurofibrillary tangles (Ghiso et al., Brain Pathol., 16:71 (2006)). Two distinct mutations in the ITM2b gene encoding BRI2 polypeptides cause FBD and FDD. The human BRI2 polypeptide is a 266 amino acid long type 2 transmembrane polypeptide of unknown function, expressed at high levels in the brain, and cleaved by furin or furin-like proteases at its carboxyl terminus to produce a 23 amino acid polypeptide (Bri23) (Kim et al., Nature Neuroscience, 2:984 (1999) and Choi et al., Faseb. J., 18:373 (2004)). Disease causing mutations result in the production of a COOH-terminally extended 277 amino acid mutant BRI2 polypeptides, which are cleaved at the normal furin processing site to generate distinct 34 amino acid polypeptides (ABri in FBD, and ADan in FDD) that accumulate in the brains of affected patients (Vidal et al., Nature, 399:776 (1999) and Vidal et al., Proc. Natl. Acad. Sci. U.S.A., 97:4920 (2000)). Notably, synthetic ABri and ADan undergo rapid aggregation and fibrillization into amyloid and, like Aβ, they are neurotoxic (Ghiso et al., Brain Pathol., 16:71 (2006) and Gibson et al., Biochem. Soc. Trans., 33:1111 (2005)). Thus, there are clear pathological and clinical similarities between FBD, FDD and Alzheimer's disease (AD). Indeed, genetic analyses of FBD, FDD, and familial forms of AD support a unifying pathologic mechanism in which accumulation of amyloidogenic peptides triggers a complex pathological cascade leading to neurodegeneration (Golde, J. Clin. Invest., 111: 11 (2003)).

### SUMMARY

This document relates to methods and materials for reducing Aβ aggregation. For example, this document provides methods and materials related to the use of BRI polypeptides (e.g., a BRI1 polypeptide, also known as integral membrane protein 2A; a BRI2 polypeptide, also known as integral membrane protein 2B; or a BRI3 polypeptide, also known as integral membrane protein 2C) and fragments of BRI polypeptides (e.g., a BRI2 polypeptide fragment such as a BRI23 polypeptide) to reduce Aβ aggregation in mammals. In addition, the methods and materials provided herein can be used to treat dementia such as (e.g., AD).

In general, one aspect of this document features a method for reducing Aβ aggregation in a mammal. The method comprises administering a composition, to the mammal, under conditions wherein Aβ aggregation in the mammal is reduced, wherein the composition comprises a BRI polypeptide or a fragment of a BRI polypeptide. The composition can comprise a Bri23 polypeptide, a Bri24 polypeptide, or a Bri25 polypeptide. The BRI polypeptide or the fragment can be unmodified. The Bri23, Bri24, or Bri25 polypeptide can comprise a D-amino acid. Each amino acid of the Bri23, Bri24, or Bri25 polypeptide can be a D-amino acid. The BRI polypeptide or the fragment can comprise one or more unnatural or modified amino acids that increase brain levels. The BRI polypeptide or the fragment can be reduced. The BRI polypeptide or the fragment can contain an intrachain disulfide bond. The composition can comprise a Bri23 polypeptide having an intrachain disulfide bond between Cys5 and Cys22. The composition can comprise a Bri24 polypeptide having an intrachain disulfide bond between Cys5 and Cys22. The composition can comprise a Bri25 polypeptide having an intrachain disulfide bond between Cys5 and Cys22.

In another aspect, this document features a method for reducing Aβ aggregation in a mammal. The method comprises administering a composition, to the mammal, under conditions wherein Aβ aggregation in the mammal is reduced, wherein the composition comprises a BRI2 polypeptide, a fragment of the BRI2 polypeptide, a nucleic acid encoding the BRI2 polypeptide, or a nucleic acid encoding the fragment. The mammal can be a human. The mammal can have Alzheimer's disease. The composition can comprise the BRI2 polypeptide. The composition can comprise the fragment. The fragment can be a Bri23 polypeptide. The composition can comprise nucleic acid encoding the BRI2 polypeptide. The composition can comprise nucleic acid encoding the fragment. The fragment can be a Bri23 polypeptide.

In another aspect, this document features a method for reducing Aβ aggregation in a mammal. The method comprises administering a composition, to the mammal, under conditions wherein Aβ aggregation in the mammal is reduced, wherein the composition comprises an agent that increases expression of a BRI polypeptide in the mammal. The composition can have the ability to increase expression of a fragment of a BRI polypeptide, wherein the fragment comprises at least 15 amino acid residues from the carboxyl terminus of a full length BRI polypeptide.

In another aspect, this document features a method for reducing Aβ aggregation in a mammal. The method comprises administering a composition, to the mammal, under conditions wherein Aβ aggregation in the mammal is reduced, wherein the composition comprises an agent that increases proteolytic cleavage of a BRI polypeptides to increase the levels of a fragment of the BRI polypeptide in the mammal. The agent can be a nucleic acid encoding a protease. The protease can be a furin protease (e.g., GenBank gi number 4505579; GenBank Accession No. NP_{—}002560.1). Other examples of proteases include, without limitation, proprotein convertase subtilisin/kexin type 2 polypeptides (e.g., GenBank gi number 56205875; GenBank Accession No. CAC34957.2), PCSK7 polypeptides (e.g., GenBank gi number 33991186; GenBank Accession No. AAH06357.1), proprotein convertase subtilisin/kexin type 4 polypeptides (e.g., GenBank gi number 76443679; GenBank Accession No. NP_{—}060043.2), proprotein convertase subtilisin/kexin type 5 polypeptides (e.g., GenBank gi number 20336246; GenBank Accession No. NP_{—}006191.2), membrane-bound transcription factor site-1 protease polypeptides (e.g., GenBank gi number 4506775; GenBank Accession No. NP_{—}003782.1), proprotein convertase subtilisin/kexin type 6 proteases (e.g., GenBank gi number 124517180; GenBank Accession No. CAM33226.1).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention arc set forth in the accompanying drawings and the description below. Other features, objects, and advantages of the invention will be apparent from the description and drawings, and from the claims.

### DESCRIPTION OF THE DRAWINGS

FIG. 1. BRI2 expression inhibits Aβ deposition in vivo. (A) Schematic of BRI2-fusion constructs. BRI2 and BRI2-Aβ1-40 are cleaved by furin and other kex2 proteases to release Bri23 and Aβ1-40, respectively. (B) P0 TgCRND8 mice were transduced by the intracerebroventricular (i.c.v.) injection of rAAV1-BRI2 or BRI2-Aβ1-40. Total brain Aβ levels (pooled values of the SDS soluble and SDS-insoluble FA extracts) from 3 months old mice were analyzed by Aβ end-specific ELISA. (C) Cortical sections of 3 months old mice were immunostained with anti-Aβ1-16 antibody 33.1.1. Sections representing the mice with the median mean levels of biochemical Aβ deposition are shown. CAA was not increased by the BRI trangenes and was almost completely absent in the TgCRND8 mice at 3 months of age. Magnification 200× (D) Amyloid plaque burdens and thioflavin positive plaques were quantified from the stitched images of whole cerebral cortex. The number of cored plaque, identified by ThioS staining, was counted individually. **P<0.01 versus no injection control (ANOVA).

FIG. 2. Comparable levels of Aβ deposition in non-injection control, PBS injection, and rAAV1-non-specific single-chain variable fragment (scFv ns) injection groups at 3 months old TgCRND8 mice. (A) P0 TgCRND8 mice were injected with PBS or rAAV1-scFv ns. Total brain Aβ levels from 3 months old mice were analyzed by Aβ end-specific ELISA. (B) Cortical sections of 3 months old mice were immunostained with anti-Aβ1-16 antibody 33.1.1 then amyloid plaque burdens were quantified from the stitched images of whole cerebral cortex sections. The extent of Aβ deposition in PBS or rAAV1-scFv ns group was comparable with non-injection control group.

FIG. 3. No evidence for alterations in APP processing or endogenous Aβ levels by expression of BRI2 and BRI2-Aβ1-40. (A) To analyze if APP processing was altered by expression of BRI2 and BRI2-Aβ1-40 in TgCNRD8 mice, SDS soluble forebrain extracts were analyzed by western blotting probed with 6E10 (anti-Aβ3-8 antibody). (B) Quantification of APP and CTFβ protein level after normalization to β-actin level showed no change in the relative levels of both proteins in all groups. (C) The steady-state endogenous mouse Aβ level in non-transgenic littermates of TgCNRD8 mice, measured by rodent Aβ-specific ELISA, were comparable between PBS injection control and rAAV1-BRI2 group.

FIG. 4. Steady state plasma levels in TgCRND8 mice and brain Aβ levels in TG2576 are not decreased by BRI2 expression. Aβ levels in plasma were analyzed by Aβ end-specific ELISA. There was no change in Aβ level in BRI2 group, compared with no injection control group. BRI2-Aβ1-40 injected TgCRND8 mice had significantly increased plasma Aβ40 level, compared with no injection and BRI2 injection group (*P<0.05). Aβ42 levels were comparable between all groups (P>0.05). (B) P0 Tg2576 mice were injected with PBS or rAAV1-BRI2 construct then the steady-state Aβ levels in 2 months old Tg2576 mice were measured by Aβ end-specific ELISA. The expression of BRI2 did not lower the steady-state Aβ levels compared with control group.

FIG. 5. BRI2-Aβ1-40 and BRI2 expression does not result in a humoral immune response to Aβ. The levels of anti-Aβ IgG antibody in plasma were measured by anti-Aβ antibody ELISA. Cerebral expression of BRI2 and BRI2-Aβ1-40 in TgCRND8 mice did not trigger anti-Aβ immune response in all groups, except in the positive control group immunized with fibrillar Aβ42.

FIG. 6. Bri23 peptide inhibits Aβ aggregation in vitro. (A) Synthetic Aβ1-42, Aβ1-40, and Bri23 peptides were mixed at the concentrations indicated and incubated at 0° C. or 37° C. for 3 hours. Following incubation the extent of Aβ aggregation into HMW aggregates was assessed by native gel electrophoresis and western blotting with Ab9 (anti-Aβ1-16) antibody that recognizes Aβ fibrils, oligomers and monomer. (B) Quantitative analysis of a second dose response study shows the difference in monomeric Aβ1-42 levels between the 37° C. and 0° C. incubations. n=3 for each condition *P<0.05 and **P<0.01 versus 1.5 μM Aβ42 aggregation (ANOVA) (C) Monomeric Aβ1-42 isolated by size exclusion chromatography was incubated in the presence or absence of Bri23 peptide. At the indicated times, aliquots of aggregation reaction mixtures were analyzed for the extent of aggregation by the ThioT assay. (D) AFM analysis of aggregates at 72 hours of incubation. Representative images, shown in height mode, are 10×10 μm and calibration bars are 1 μm.

FIG. 7. The Bri23 peptide is required for the anti-amyloidogenic effect of the BRI2 protein in vivo. (A) Schematic of BRI2 and BRI2del244-266 constructs. BRI2del244-266 construct does not encode the Bri23 peptide. P0 TgCRND8 mice were transduced by i.c.v. injection of rAAV1-BRI2del244-266. (B) Cortical sections (magnification 200×) of 3 months old TgCRND8 mice were immunostained with anti-Aβ1-16 antibody (33.11) and amyloid plaque burdens were quantified (C). (D) Total brain Aβ levels were analyzed by Aβ end-specific ELISA after 3 months of post-transduction. (E) Western blot analysis of steady state levels of the rAAV1 delivered trangenes. BRI2, BRI2-Aβ1-40 and BRI2del244-266 all migrate at ˜37 kDa. Anti-β actin is used as a loading control.

FIG. 8. Genetic Association of ITM2b haplotypes with AD and detection of Bri23 in human CSF. (A) All subjects. (B) Subjects with ages at diagnosis/entry of 60-80 years. (C) Subjects with ages at diagnosis/entry of 80-103 years. Haplotypes were identified using the expectation maximization algorithm implemented in Haplo Stats. Global p values were obtained using the score statistic implemented in Haplo Stats. Odds ratios and 95% confidence intervals show each haplotype compared to all others and were obtained by univariable logistic regression using gender, age at diagnosis/entry, and ApoE ε4 (+/−) genotype as covariates. (D) Bri23 was detected by HPLC/MS in conditioned media from H4 cells transiently transfected with BRI2 in a pCDNA3 expression vector. Bri23 is not detected in H4 cells transiently transfected with pcDNA3 BRI2-del244-266. Standard refers to synthetic Bri23 (Bachem). (E) HPLC/MS detection of Bri23 in human CSF. HPLC analysis of 50 μL of human CSF demonstrates identifies peptides in the CSF of three patients with AD (age and sex indicated in each panel) with identical mass that elute in the same HPLC fraction as synthetic Bri23 is consistent. The assessed mass of synthetic Bri23 is 2630.42, which is in good agreement with its predicted mass of 2629.99. Values obtained for peaks in the individual samples are within 1 Da of the Bri23 standard.

FIG. 9. Association of ITM2B multilocus genotypes (MLGs) with AD and ITM2B mRNA levels. (A) Association of the sets of risky, intermediate, and protective multilocus genotypes with AD. The OR and 95% confidence interval for each set of multilocus genotypes compared to all others was determined by logistic regression using gender, age at diagnosis/entry, and ApoE ε4 (+/−) as covariates. Black symbols show the exploratory (JS) series, red symbols show the follow-up (RS-AUT) series, and green symbols show the combined series. In panel B, the four genotypes comprising the risky group are shaded pink, the nine comprising the intermediate risk group are shaded gray, and the two comprising the protective (low risk) group are shaded green (B). Bar charts showing the percentage of all 60-80 year old AD and control subjects with each of the 15 MLG groups (haplotype pairs) shown in Table 3. The haplotype pairs corresponding to each MLG are arranged as in Table 3 with high risk odds ratios at the bottom of each column and low risk (protective) odds ratios at the top. The H1/H5 (p=0.039), H2/H2 (p=0.050), H1/H6 (p=0.051), and H2/H5 (p=0.14) genotypes shown in pink form a high risk group with significant or suggestive association, the H1/H4 (p=0.023) and H1/H8 (p=0.14) genotypes shown in green form a low risk group with significant or suggestive association, and the remaining nine genotypes (0.35<p<0.97) form an intermediate group which did not show significant or suggestive association (Table 3). (C) Association of ITM2B multilocus genotypes with ITM2B mRNA levels. Using real time PCR with 18s RNA as reference, ITM2B mRNA was analyzed in the cerebellum of 141 AD brains. ITM2B mRNA levels were significantly increased by 32% (p=0.02 by two sided Mann Whitney test) in the 116 subjects with any of the eleven low risk genotypes as compared to the 25 subjects with any of the four high risk genotypes (H1/H5, H2/H2, H1/H6, H2/H5). In these box and whisker plots, the central box encompasses values with ranks in the second and third quartiles, the diamond shows the median value and the whiskers extend from the minimum to maximum values.

FIG. 10. Oxidized BRI polypeptides inhibit Aβ42 aggregation in vitro. The oxidized forms of Bri24 (Bri1-24), Bri23 (Bri2-23), and Bri25 (Bri3-25) inhibit Aβ42 aggregation in vitro. BRI polypeptides in DMSO were diluted into 150 mM NaCl, 20 mM Tris-HCl, pH7.4 (TBS) at a final concentration of 2 μM in the absence (OX) or presence (RED) of 2 mM DTT. Mixtures were incubated for 10 minutes at room temperature and then chilled to 0° C. A control samples lacking any BRI polypeptide were prepared identically using DMSO alone. Aβ42 in DMSO then was added to a final concentration of 1.5 μM, and the mixtures divided into two aliquots. One aliquot was incubated for 5 hours at 0° C., and the other was incubated at 37° C. to induce Aβ42 aggregation. At the end of the incubation, all samples were returned to 0° C. to stop further aggregation prior to analysis. Aggregation was assessed either by native gel electrophoresis followed by western blotting (panel A) or by the ELISA-based assay (panel B).

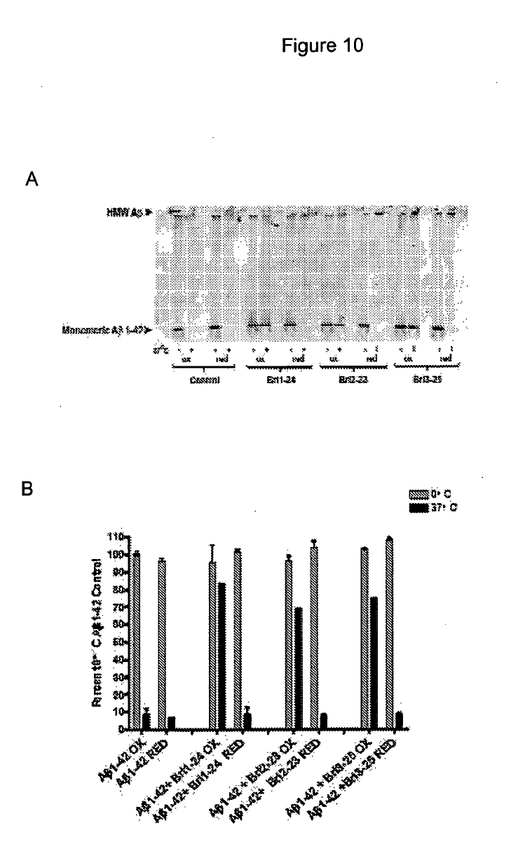

### DETAILED DESCRIPTION

This document relates to methods and materials for reducing Aβ aggregation. For example, this document provides methods and materials related to the use of BRI polypeptides (e.g., BRI1 polypeptides, BRI2 polypeptides, or BRI3 polypeptides) and fragments of BRI polypeptides (e.g., a Bri23 polypeptide, a Bri24 polypeptide, or a Bri25 polypeptide) to reduce Aβ aggregation in mammals. In addition, the methods and materials provided herein can be used to treat dementia such as (e.g., AD).

This document provides BRI polypeptides, polypeptide fragments of a BRI polypeptide (e.g., a Bri23 polypeptide), and methods for making and using such polypeptides and polypeptide fragments. An BRI polypeptide (e.g., a BRI1 polypeptide, a BRI2 polypeptide, or a BRI3 polypeptide) can be from any species including, without limitation, dogs, cats, horses, bovine, sheep, monkeys, and humans. Amino acid sequences for BRI1 polypeptides can be as set forth in GenBank gi accession numbers 149055528, 51556454, and 48145867 (see, also, accession numbers EDM07112, NP_{—}032435, and CAG33156). A human BRI1 polypeptide can have the following amino acid sequence: MVKIAFNTPTAVQKEEARQDVEALLSRTV-RTQILTGKELRVATQEKEGSSGRCMLTLLGLSFILAGLIVGGACIYKYFMPKSTIY RGEMCFFDSEDPANSLRGGEPNFLPVTEEADIREDDNIAIIDVPVPSFSDSDPAAII HDFEKGMTAYLDLLLGNCYLMPLNTSIVMPPKNLVELFGKLASGRYLPQTYVVR EDLVAVEEIRDVSNLGIFIYQLCNNRKSFRLRRRDLLLGFNKRAIDKCWKIRHFPN EFIVETKICQD (SEQ ID NO:1). Nucleic acid sequences that encode a BRI1 polypeptide can be as set forth in GenBank gi accession numbers 51556453, 71043803, and 74316000 (sec, also, accession numbers NM_{—}008409, NM_{—}001025712, and NM_{—}004867).

Amino acid sequences for BRI2 polypeptides can be as set forth in GenBank gi accession numbers 6680502, 55661804, and 55741681 (see, also, accession numbers NP_{—}032436, CAH71157.1, and NP_{—}001006964). A human BRI2 polypeptide can have the following amino acid sequence: MVKVTFNSALAQKEAKKDEPKSGEEALI-IPPDAVAVDCKDPDDVVPVGQRRAWCWCMCFGLAFMLAGVILGGAYLYKYFA LQAGTYLPQSYLIHEHMVITDRIENIDHLGFFIYRLCHDKETYKLQRRETIKGIQK REASNCFAIRHFENKFAVETLICS (SEQ ID NO:2). Nucleic acid sequences that encode a BRI2 polypeptide can be as set forth in GenBank gi accession numbers 133892559, 142388886, and 55741680 (see, also, accession numbers NM_{—}008410, NM_{—}021999, and NM_{—}001006963).

Amino acid sequences for BRI3 polypeptides can be as set forth in GenBank gi accession numbers 11967943, 149016317, and 48146533 (see, also, accession numbers NP_{—}071862, EDL75563, and CAG33489). A human BRI3 polypeptide can have the following amino acid sequence: MVKISFQPAVAGIKGDKADKASASAPAPASA-TEILLTPAREEQPPQHRSKRGSSVGGVCYLSMGMVVLLMGLVFASVYIYRYFFL AQLARDNFFRCGVLYEDSLSSQVRTQMELEEDVKIYLDENYERINVPVPQFGGG DPADIIHDFQRGLTAYHDISLDKCYVIELNTTIVLPPRNFWELLMNVKRGTYLPQT YIIQEEMVVAEHVSDKEALGSFIYHLCNGKDTYRLRRRATRRRINKRGAKNCNAI RHFENTFVVETLICGVV (SEQ ID NO:3). Nucleic acid sequences that encode a BRI3 polypeptide can be as set forth in GenBank gi accession numbers 142386544, 57527253, and 60302915 (see, also, accession numbers NM_{—}022417, NM_{—}001009674, and NM_{—}030926).

A polypeptide fragment of a BRI polypeptide can be any length greater than 5 amino acid residues and can contain the following core sequence FxxxF (e.g., FEGKF). In some cases, a polypeptide fragment of a BRI polypeptide can contain at least 6, 7, 8, 9, 10, 11, 12, 13, 14, 15, 16, 17, 18, 19, 20, 21, 22, 23, 24, or 25 amino acids from the C-terminus of a full-length BRI polypeptide. For example, a BRI polypeptide fragment can contain the 20 C-terminal amino acid residues of the amino acid sequence set forth in SEQ ID NO:2. In some cases, a fragment of a BRI polypeptide can be between 15 amino acid residues and 100 amino acid residues (e.g., between 10 and 50 amino acid residues, between 15 and 50 amino acid residues, between 20 and 50 amino acid residues, between 20 and 40 amino acid residues, between 20 and 30 amino acid residues, or between 20 and 25 amino acid residues). Examples of such fragments include the polypeptides set forth in Table 1.

TABLE 1
Fragments of BRI polypeptides.

| Name of | |
|---|---|
| Polypeptide | Amino Acid Sequence |
| Bri23 (ITM2B) | EASNCFAIRHFENKFAVETLICS |
| | (SEQ ID NO: 4) |
| Bri24 (ITM2A) | AIDKCWKIRHFPNEFIVETICICQD |
| | (SEQ ID NO: 5) |
| Bri25 (ITM2C) | GAICNCNAIRHFENTFVVETLICGVV |
| | (SEQ ID NO: 6) |

A polypeptide fragment of a BRI2 polypeptide can lack an amino acid sequence set forth in Table 2. For example, a BRI2 polypeptide fragment can have the amino acid sequence set forth in SEQ ID NO:4 and lack the amino acid sequence set forth in any of SEQ ID NOs:7-9.

TABLE 2
Amino acid sequences.

| SEQ | |
|---|---|
| ID | |
| NO: | Amino Acid Sequence |
| 7 | MVKVTFNSALAQKEAKKDEPKSGEEALIIPPDAVAVDCKDPDDVVPVG |
| | QRRAWCWCMCFGLAFMLAGVILGGAYLYKYFALQAGTYLPQSYLIHE |
| | HMVITDRIENIDHLGFFIYRLCHDKETYKLQRRETIKGIQKR |
| 8 | MVKVTFNSALAQKEAKKDEPKSGEEALIIPPDAVAVDCKDPDDVVPVG |
| | QRRAWCWCMCFGLAFMLAGVILGGAYLYKYFALQAGTYLPQSYLIHE |
| | HMVITDRIENIDHLGFFIYRLCHDKETYKLQRR |
| 9 | MVKVTFNSALAQKEAKKDEPKSGEEALIIPPDAVAVDCKDPDDVVPVG |
| | QRRAWCWCMCFGLAFMLAGVILGGAYLYKYFALQAGTYLPQSYLIHE |
| | HMVITDRIENIDHL |

The polypeptides and polypeptide fragments provided herein can be substantially pure. The term “substantially pure” as used herein with reference to a polypeptide means the polypeptide is substantially free of other polypeptides, lipids, carbohydrates, and nucleic acid with which it is naturally associated. A substantially pure polypeptide can be any polypeptide that is removed from its natural environment and is at least 60 percent pure. A substantially pure polypeptide can be at least about 65, 70, 75, 80, 85, 90, 95, or 99 percent pure. Typically, a substantially pure polypeptide will yield a single major band on a non-reducing polyacrylamide gel. A substantially pure polypeptide can be a chemically synthesized polypeptide.

Any method can be used to obtain a substantially pure polypeptide provided herein. For example, common polypeptide purification techniques such as affinity chromotography and HPLC as well as polypeptide synthesis techniques can be used to obtain a BRI2 polypeptide or fragment thereof. In addition, any material can be used as a source to obtain a substantially pure polypeptide. In some cases, tissue culture cells engineered to over-express a particular polypeptide can be used to obtain substantially pure polypeptide. Further, a polypeptide can be engineered to contain an amino acid sequence that allows the polypeptide to be captured onto an affinity matrix. For example, a tag such as c-myc, hemagglutinin, polyhistidine, or Flag™ tag (Kodak) can be used to aid polypeptide purification. Such tags can be inserted anywhere within the polypeptide including at either the carboxyl or amino termini, or in between. Other fusions that can be used include enzymes that aid in the detection of the polypeptide, such as alkaline phosphatase.

A BRI polypeptide or polypeptide fragment provided herein can contain one or more modifications. For example, a BRI2 polypeptide or fragment thereof can be modified to be pegylated or acylated. In some cases, a BRI polypeptide or fragment thereof can be covalently attached to oligomers, such as short, amphiphilic oligomers that enable oral administration or improve the pharmacokinetic or pharmacodynamic profile of a conjugated BRI polypeptide or fragment thereof. The oligomers can comprise water soluble PEG (polyethylene glycol) and lipid soluble alkyls (short chain fatty acid polymers). See, for example, International Patent Application Publication No. WO 2004/047871. In some cases, a BRI polypeptide or fragment thereof can be fused to the Fc domain of an immunoglobulin molecule (e.g., an IgG1 molecule) such that active transport of the fusion polypeptide across epithelial cell bathers via the Fc receptor occurs. In some cases, a polypeptide provided herein can contain chemical structures such as ε-aminohexanoic acid; hydroxylated amino acids such as 3-hydroxyproline, 4-hydroxyproline, (5R)-5-hydroxy-L-lysine, allo-hydroxylysine, and 5-hydroxy-L-norvaline; or glycosylated amino acids such as amino acids containing monosaccharides (e.g., D-glucose, D-galactose, D-mannose, D-glucosamine, and D-galactosamine) or combinations of monosaccharides. In some cases, a polypeptide provided herein such as a polypeptide fragments provided herein (e.g., a Bri23 polypeptide) can be a cyclic polypeptide.

A polypeptide provided herein can contain one or more amino acid additions, subtractions, or substitutions relative to another polypeptide (e.g., a wild-type BRI2 polypeptide or fragment thereof). Such polypeptides can be prepared and modified as described herein. Amino acid substitutions can be conservative amino acid substitutions. Conservative amino acid substitutions are, for example, aspartic-glutamic as acidic amino acids; lysine/arginine/histidine as basic amino acids; leucine/isoleucine, methionine/valine, alanine/valine as hydrophobic amino acids; serine/glycine/alanine/threonine as hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine.

In some cases, amino acid substitutions can be substitutions that do not differ significantly in their effect on maintaining (a) the structure of the polypeptide backbone in the area of the substitution, (b) the charge or hydrophobicity of the molecule at the target site, or (c) the bulk of the side chain. Naturally occurring residues are divided into groups based on common side-chain properties: (1) hydrophobic: norleucine, met, ala, val, lcu, ile; (2) neutral hydrophilic: cys, ser, thr; (3) acidic: asp, glu; (4) basic: asn, gln, his, lys, arg; (5) residues that influence chain orientation: gly, pro; and (6) aromatic; trp, tyr, phe. In some cases, non-conservative substitutions can be used. A non-conservative substitution can include exchanging a member of one of the classes described herein for another.

Any route of administration (e.g., oral or parenteral administration) can be used to administer a polypeptide or composition provided herein (e.g., a composition containing one or more of the polypeptides provided herein) to a mammal. For example, a composition can be administered orally or parenterally (e.g., a subcutaneous, intramuscular, intraorbital, intracapsular, intraspinal, intrasternal, intracranial, or intravenous injection). Compositions containing a polypeptide provided herein can contain additional ingredients such as those described in U.S. Pat. No. 6,818,619. Such additional ingredients can be polypeptides or non-polypeptides (e.g., buffers). In addition, the polypeptides within a composition provided herein can be in any form such as those described in U.S. Pat. No. 6,818,619.

In some cases, a nucleic acid encoding a BRI polypeptide or a fragment thereof can be administered to a mammal to reduce Aβ aggregation or to treat dementia. Such a nucleic acid can encode a full-length BRI polypeptide (e.g., a BRI2 polypeptide such as a human BRI2 polypeptide having the amino acid sequence set forth in SEQ ID NO:2 or a fragment of a BRI2 polypeptide (e.g., a Bri23 polypeptide)). A nucleic acid encoding a BRI polypeptide or a fragment thereof can be administered to a mammal using any appropriate method. For example, a nucleic acid can be administered to a mammal using a vector such as a viral vector.

Vectors for administering nucleic acids (e.g., a nucleic acid encoding a BRI2 polypeptide) to a mammal are known in the art and can be prepared using standard materials (e.g., packaging cell lines, helper viruses, and vector constructs). See, for example, Gene Therapy Protocols (Methods in Molecular Medicine), edited by Jeffrey R. Morgan, Humana Press, Totowa, N.J. (2002) and Viral Vectors for Gene Therapy: Methods and Protocols, edited by Curtis A. Machida, Humana Press, Totowa, N.J. (2003). Virus-based nucleic acid delivery vectors are typically derived from animal viruses, such as adenoviruses, adeno-associated viruses, retroviruses, lentiviruses, vaccinia viruses, herpes viruses, and papilloma viruses.

Lentiviruses are a genus of retroviruses that can be used to infect neuronal cells and non-dividing cells. Adenoviruses contain a linear double-stranded DNA genome that can be engineered to inactivate the ability of the virus to replicate in the normal lytic life cycle. Adenoviruses can be used to infect dividing and non-dividing cells. Adenoviral vectors can be introduced and efficiently expressed in cerebrospinal fluid and in brain. Adeno-associated viruses also can be used to infect non-dividing cells. Muscle cells and neurons can be efficient targets for nucleic acid delivery by adeno-associated viruses. Additional examples of viruses that can be used as viral vectors include herpes simplex virus type 1 (HSV-1). HSV-1 can be used as a neuronal gene delivery vector to establish a lifelong latent infection in neurons. HSV-1 can package large amounts of foreign DNA (up to about 30-40 kb). The HSV latency-associated promoter can be used to allow high levels of expression of nucleic acids during periods of viral latency.

Vectors for nucleic acid delivery can be genetically modified such that the pathogenicity of the virus is altered or removed. The genome of a virus can be modified to increase infectivity and/or to accommodate packaging of a nucleic acid, such as a nucleic acid encoding a BRI2 polypeptide. A viral vector can be replication-competent or replication-defective, and can contain fewer viral genes than a corresponding wild-type virus or no viral genes at all.

In addition to nucleic acid encoding a BRI polypeptide or a fragment thereof, a viral vector can contain regulatory elements operably linked to a nucleic acid encoding a BRI polypeptide or a fragment thereof. Such regulatory elements can include promoter sequences, enhancer sequences, response elements, signal peptides, internal ribosome entry sequences, polyadenylation signals, terminators, or inducible elements that modulate expression (e.g., transcription or translation) of a nucleic acid. The choice of element(s) that may be included in a viral vector depends on several factors, including, without limitation, inducibility, targeting, and the level of expression desired. For example, a promoter can be included in a viral vector to facilitate transcription of a nucleic acid encoding a BRI polypeptide or a fragment thereof. A promoter can be constitutive or inducible (e.g., in the presence of tetracycline), and can affect the expression of a nucleic acid encoding a BRI polypeptide or a fragment thereof in a general or tissue-specific manner. Tissue-specific promoters include, without limitation, enolase promoter, prion protein (PrP) promoter, and tyrosine hydroxylase promoter.

As used herein, “operably linked” refers to positioning of a regulatory element in a vector relative to a nucleic acid in such a way as to permit or facilitate expression of the encoded polypeptide. For example, a viral vector can contain a neuronal-specific enolase promoter and a nucleic acid encoding a BRI2 polypeptide or a fragment thereof. In this case, the enolase promoter is operably linked to a nucleic acid encoding a BRI2 polypeptide or a fragment thereof such that it drives transcription in neuronal tissues.

A nucleic acid encoding a BRI polypeptide or a fragment thereof also can be administered to a mammal using non-viral vectors. Methods of using non-viral vectors for nucleic acid delivery are known to those of ordinary skill in the art. See, for example, Gene Therapy Protocols (Methods in Molecular Medicine), edited by Jeffrey R. Morgan, Humana Press, Totowa, N.J. (2002). For example, a nucleic acid encoding a BRI2 polypeptide or a fragment thereof can be administered to a mammal by direct injection of nucleic acid molecules (e.g., plasmids) comprising nucleic acid encoding a BRI2 polypeptide or a fragment thereof, or by administering nucleic acid molecules complexed with lipids, polymers, or nanospheres.

A nucleic acid encoding a BRI polypeptide or a fragment thereof can be produced by standard techniques, including, without limitation, common molecular cloning, polymerase chain reaction (PCR), chemical nucleic acid synthesis techniques, and combinations of such techniques. For example PCR or RT-PCR can be used with oligonucleotide primers designed to amplify nucleic acid (e.g., genomic DNA or RNA) encoding a BRI2 polypeptide or a fragment thereof.

The invention will be further described in the following examples, which do not limit the scope of the invention described in the claims.

### Examples

### Example 1

### BRI2 Inhibits Aβ Deposition in vivo and Shows Genetic Association with Alzheimer's Disease

rAAV1 Construction and Preparation

rAAV1 expressing BRI2, BRI2-Aβ1-40, BRI2del244-266, non-specific single-chain variable fragment (scFv ns), or enhanced green fluorescent protein (eGFP), under the control of the cytomegalovirus enhancer/chicken β actin (CBA) promoter were generated by calcium-phosphate transfection of pAM/CBA-pI-WPRE-BGH, rAAV1 cis plasmid pH21 (AAV1 helper plasmid) and pFΔ6 into a HEK293 cell line. rAAV1-scFv ns construct is described elsewhere (Levites et al., J. Neurosci. 26:11923 (2006)). At 48 hours after transfection, cells were lysed in the presence of 0.5% sodium deoxycholate and 50 U/mL benzonase (Sigma) by repeated rounds of freeze/thaws at −80° C. and −20° C. The virus was isolated using a discontinuous Iodixanol gradient, and then affinity purified on a HiTrap HQ column (Amersham). Samples were eluted from the column, and the buffer exchanged to PBS using an Amicon Ultra 100 Centrifugation device (Millipore). The genomic titer of each virus was determined by quantitative PCR using the ABI 7900 (Applied Biosystems). The viral DNA samples were prepared by treating the virus with DNaseI (Invitrogen), heat inactivating the enzyme, then digesting the protein coat with Proteinase K (Invitrogen), followed by a second heat-inactivation. Samples were compared against a standard curve of supercoiled plasmid.

rAAV1 Injection to Neonatal Mice

TgCRND8 mice expressing mutant human APP (KM670/671NL and V717F) gene under the control of hamster prion promoter are described elsewhere (Chishti et al., J. Biol. Chem. 276:21562 (2001)). Hemizygous male TgCRND8 mice were crossed with female B6C3F1 wild-type mice. Tg2576 mice expressing mutant human APP (KM670/671NL) gene under the control of hamster prion promoter are described elsewhere (Hsiao et al., Science, 274:99 (1996) and Passini et al., J. Virol., 77:7034 (2003)). Hemizygous female Tg2576 mice were mated with male B6SJL wild-type mice. The injection procedures were performed as described elsewhere (Levites et al., J. Neurosci., 26:11923 (2006); Passini et al., J. Virol., 77:7034 (2003); and Broekman et al., Neuroscience, 138:501 (2006)). Briefly, P0 pups were cryoanesthetized on ice for 5 minutes. Two μL of AAV1 construct (1×10¹² genome particles/mL) was bilaterally injected into the cerebral ventricle of newborn mice using a 10 mL Hamilton syringe with a 30 gauge needle. The pups were placed on a heating pad until they recovered from cryoanesthesia then returned to their mother for further recovery. Negative control groups (total n=20) were non-injection (n=4), PBS injection (n=4), eGFP (n=5) and non-specific scFv (n=7) groups. Experimental groups were BRI2-Aβ1-40 (n=11), BRI2 (n=8), and BRI2del244-266 (n=13). Biochemical and histochemical Aβ loads in the control groups were equivalent.

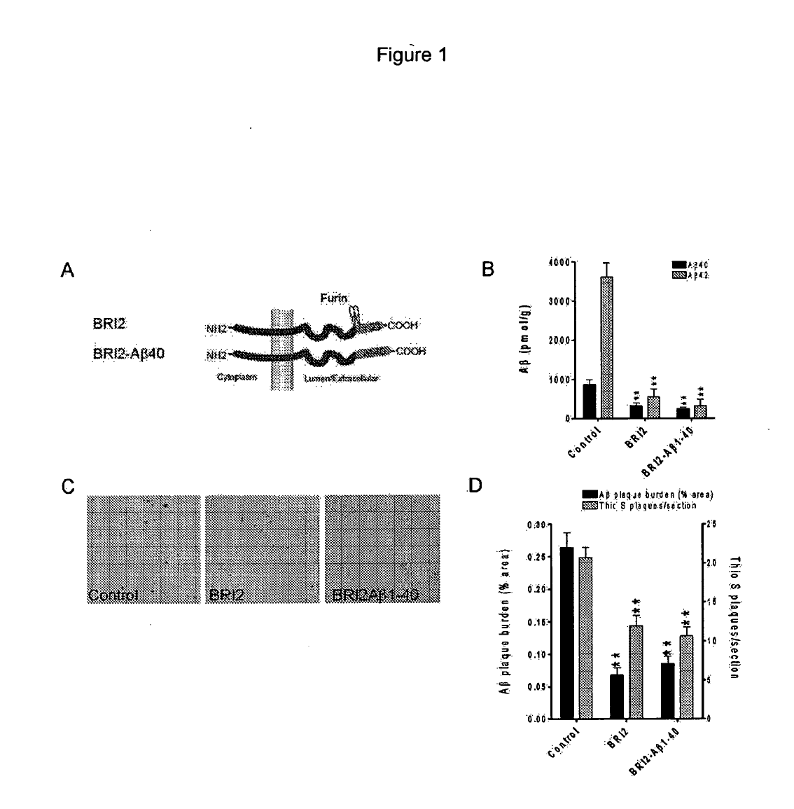

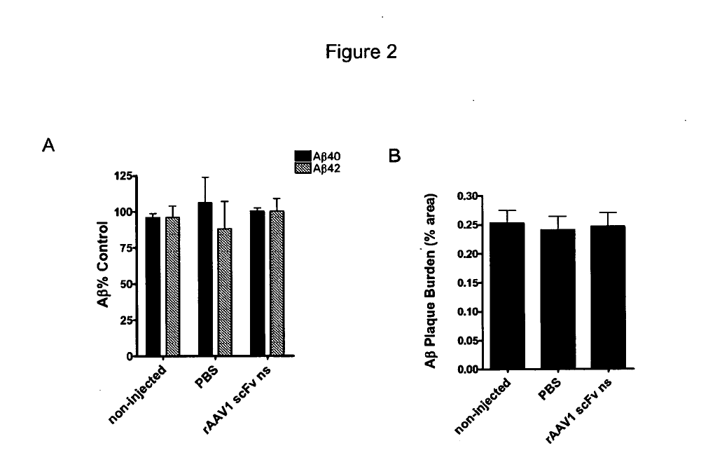

The effects of the virally delivered BRI2-Aβ1-40 transgene were compared to effects of the rAAV1 delivered human BRI2 transgene and a non-injection control (FIG. 1A). Expression of BRI2 was intended to serve as a second control, as rAAV1 delivery or mock virus delivery did not alter Aβ deposition (FIG. 2) (Levites et al., J. Neurosci., 26:11923 (2006)). Three months after rAAV1 mediated transgene delivery, mice were killed and brain Aβ deposition was analyzed using both biochemical and histochemical methods. These analyses revealed a dramatic suppressive effect of both the BRI2-Aβ1-40 and BRI2 transgenes on parenchymal Aβ accumulation (FIG. 1B-D).

### Quantification of Amyloid Deposition

Hemibrains were immersion fixed in 10% formalin then processed for paraffin embedding. Brain tissue sections (5 μm) were immunostained with the anti-total Aβ antibody (33.1.1, 1:1000) on a DAKO autostainer. The cortical Aβ plaque burden and the number of ThioS positive plaques were quantified as described elsewhere (Kim et al., J. Neurosci., 27:627 (2007)).

### Aβ Sandwich ELISA

For brain Aβ ELISAs from TgCRND8 mice, hemi-forebrains were homogenized in 2% SDS with 1× protease inhibitor cocktail (Roche) dissolved in H2O then ultra-centrifuged at 100,000 g for 1 hour. The SDS-insoluble Aβ were extracted using 70% formic acid (FA). For brain Aβ ELISAs from 2 months old Tg2576 mice, hemi-forebrains were homogenized in radioimmunoprecipitation assay (RIPA) buffer (0.1% SDS, 0.5% Deoxycholate, 1% Triton X-100, 150 mM NaCl, and 50 mM Tris-HCl) then ultracentrifugcd at 100,000 g for 1 hour. To measure the endogenous mouse Aβ levels, hemi-forebrains of non-transgenic littermates of the TgCRND8 mice expressing BRI2 were homogenized in 0.2% diethylamine (DEA) buffer containing 50 mM NaCl and 1× protease inhibitor cocktail (Roche). Endogenous mouse Aβ levels were measured using the previous validated rodent specific Aβ ELISA system as described elsewhere (Eckman et al., J. Biol. Chem., 281:30471 (2006)). For plasma Aβ analysis, blood was collected in EDTA-coated tubes following cardiac puncture. Blood samples were centrifuged at 3000 rpm for 10 minutes at 4° C. and then the plasma was aliquoted and stored at −80° C. until used. Aβ levels were determined by human Aβ end-specific sandwich ELISAs as described elsewhere (Kim et al., J. Neurosci., 27:627 (2007)).

### Mouse Anti-Aβ IgG ELISA

To test whether mice generate anti-Aβ antibody responses, anti-Aβ IgG antibody titers were determined by standard ELISA techniques, as described elsewhere (Das et al., Neurobiol. Aging, 22:721 (2001)). Briefly, microtitre plates (Maxi Sorp, Dynatech) were coated with aggregated Aβ42 at 2 μg/well. After washings, serial dilutions of plasma (1:500 dilution) were added and incubated overnight at 4° C. Following washes with PBS/0.1% Tween-20, plasma IgG was detected using a anti-mouse IgG antibody conjugated with HRP (1:2000, Sigma) and TMB substrate (KPL).

### Western Blotting

Snap frozen forebrain samples were homogenized in 2% SDS buffer with 1× protease inhibitor cocktail (Roche). The homogenate was centrifuged at 100,000 g for 1 hour at 4° C. Protein concentration in supernatants was determined using the BCA Protein Assay kit (Pierce). Protein samples (20 μg) were separated on Bis-Tris 12% XT gels (Biorad) with XT-MES buffer or Bis-Tris 4-12% XT gels (Biorad) with XT-MOPS buffer and transferred to 0.2 μm nitrocellose membranes. Blots were microwaved for 2 minutes in 0.1 M PBS twice and probed with the antibody 82E1 (anti-Aβ1-16, 1:1000, IBL), CT20 (anti-APP C-terminal 20 amino acids, 1:1000) and ITM2b (GenWay). Blots were stripped and reprobed with anti β-actin (1:1000, Sigma) as a loading control. Relative band intensity was quantified using ImageJ software (NIH).

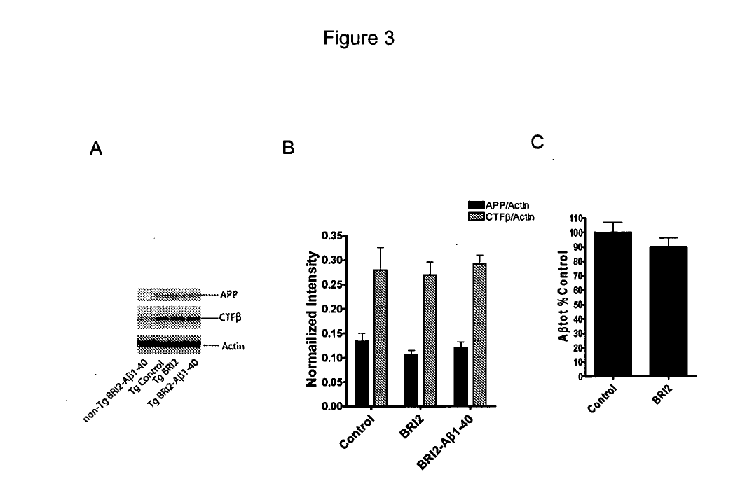

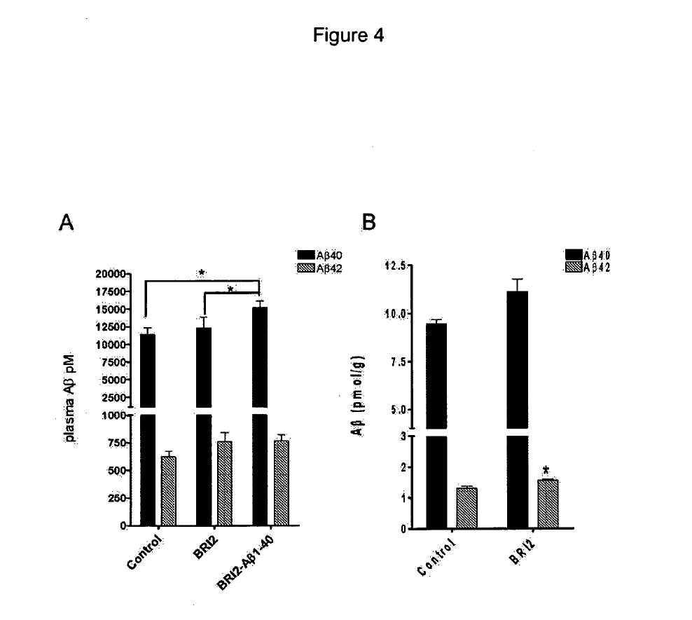

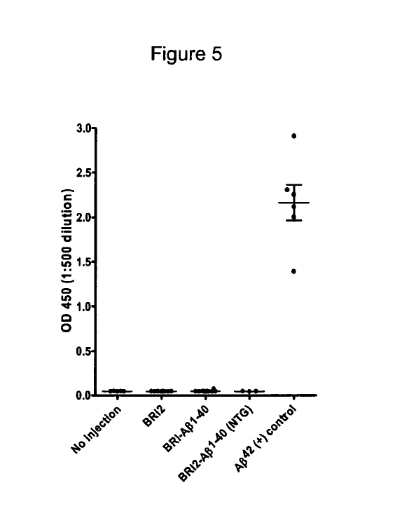

The reduction in Aβ deposition observed in the mice expressing the rAAV1 BRI2-Aβ1-40 transgenes was entirely consistent with transgenic mice studies described elsewhere (Kim et al., J. Neurosci., 27: 627 (2007)); however, the reduction of Aβ deposition observed in the mice expressing BRI2 was unexpected. A potential interaction between BRI2 and APP and noted that BRI2 overexpression increased APP CTFβ and reduced Aβ secretion in cultured cells is described elsewhere (Fotinopoulou et al., J. Biol. Chem., 280:30768 (2005) and Matsuda et al., J. Biol. Chem., 280:28912 (2005)). No evidence for alterations in the steady state levels of APP or APP CTFβ in TgCRND8 mice expressing the virally delivered BRI2-Aβ1-40 or BRI2 transgenes was found (FIG. 3). Analyses of endogenous rodent Aβ levels in the brains of the non-transgenic littermates of the TgCRND8 mice expressing the BRI2 transgene revealed that steady state Aβ levels were not affected by BRI2 expression (FIG. 3). BRI2-Aβ1-40 expression slightly increased plasma Aβ40 levels, attributable to brain to plasma efflux of Aβ1-40; plasma Aβ1-40 levels were not significantly changed by BRI2 expression (FIG. 4). In addition, rAAV1 mediated delivery of the BRI2 to P0 Tg2576, a mouse model in which Aβ deposition begins at 6-8 months, did not lower steady state brain Aβ levels in brains of 2 month old mice (FIG. 4). As anti-Aβ antibodies reduce Aβ deposition in mice and expression of virally encoded Aβ peptides in the periphery has been shown to generate an anti-Aβ response, whether central nervous system (CNS) delivery of the transgene induced a humoral immune response to Aβ was examined. There was no evidence for an anti-Aβ titer in any of the rAAV1 injected mice (FIG. 5). These results demonstrate that the reduction of Aβ accumulation by BRI2 and BRI2-Aβ1-40 transgenes was not attributable to alterations in APP processing or induction of an anti-Aβ immune response.

In vitro Aβ Aggregation Assay Using Native Gel Electrophoresis

To understand the underlying mechanism by which BRI2 reduced Aβ accumulation, whether Bri23 polypeptide could directly inhibit Aβ1-42 in vitro fibrillogenesis was tested. Synthetic Aβ1-42 and Aβ1-40, treated with HFIP and dried (Bachem), and Bri23 polypeptides (Bachem) were dissolved in DMSO then diluted in TBS at molar ratios as indicated. Aβ1-42 and Bri23 peptide mixtures were either incubated for 3 hours at 0° C. or 37° C. without shaking. Mixtures were separated on 4-20% Tris-HCl gels under nondenaturing conditions and transferred to 0.4 μm PVDF membrane as described elsewhere (Kim et al., J. Neurosci., 27:627 (2007) and Klug et al., Eur. J. Biochem., 270:4282 (2003)). The blot was probed with Ab9 (anti-Aβ1-16, 1:1000). Relative band intensity was quantified using ImageJ software (NIH).

In vitro Aβ1-42 Aggregation Assay Using Thioflavin T and AFM Studies.

Bri23 polypeptides (Bachem) were reconstituted in 1 mg/mL Tris-HCl (pH 8.0). The lyophilized synthetic Aβ1-42 (Mayo Clinic Peptide Synthesis Facility) was dissolved at 0.5-2.0 mM in 20 mM NaOH 15 minutes prior to size exclusion chromatography on Superdex 75 HR 10/30 column (Amersham Pharmacia) to remove any pre-formed Aβ aggregates. The concentration of monomeric Aβ was determined by UV absorbance with a calculated extinction coefficient of 1450 cm⁻¹M⁻¹ at 276 nm (Rangachari et al., Biochemistry, 45:8639 (2006)). Aβ1-42 aggregation reactions were initiated in siliconized eppendorf tubes by incubating 25-50 μM of freshly purified Aβ1-42 monomer in 10 mM Tris-HCl and 150 mM NaCl (pH 8.0) buffer without agitation at 37° C. Monomeric Aβ1-42 aggregation process in the presence or absence of Bri23 polypeptide were monitored using a thioflavin T assay as described elsewhere (Rangachari et al., Biochemistry, 45:8639 (2006)). Atomic force microscopy images were obtained with a NanoScope III controller with a Multimode AFM (Veeco Instruments Inc, Chadds Ford Pa.) as described elsewhere (Nichols et al., J. Biol. Chem., 280:2471 (2005)). Images are shown in amplitude mode, where increasing brightness indicates greater damping of cantilever oscillation.

### HPLC/MS Analysis of Bri23 Polypeptides.

Conditioned media or CSF was filtered through a 0.45 μM syringe filter to remove large particulate matter. A fifty microliter aliquot of the sample was injected into an Agilent 1100 Series HPLC with a Zobax Eclipse XDB-C8 column and running buffer of acetonitrile/H2O (ACN:H2O) with 0.1% trifluoroacetic acid (TFA) at a flow rate of one milliliter a minute. Initial solvent composition was 20:80 ACN/H2O, this composition was held for three minutes then linearly ramped up to 37:63 ACN/H2O over the next seven minutes. A fraction was collected between 9.4 minutes and 10.4 minutes (as the BRI-23 standard was seen to elute at 9.8 minutes) for a total of one milliliter. The collected fraction was then blown down in nitrogen at 37° C. to approximately 100 μL in volume. A one microliter aliquot of this concentrated sample was applied to a Bio-Rad gold array chip and allowed to air dry. After the sampled dried, one microliter of saturated α-Cyano-4-hydroxycinnamic acid (MALDI matrix) in 70:20:10 ACN:H2O:MeOH w/0.1% TFA was applied on top of dried sample and allowed to air dry. This was then analyzed on a Bio-Rad Ciphergen ProteinChip SELDI time-of-flight system. A laser intensity of 750 ∥J was used to collect spectra from 3975 laser shots which were averaged into the final spectra. The finished spectra were baseline corrected.

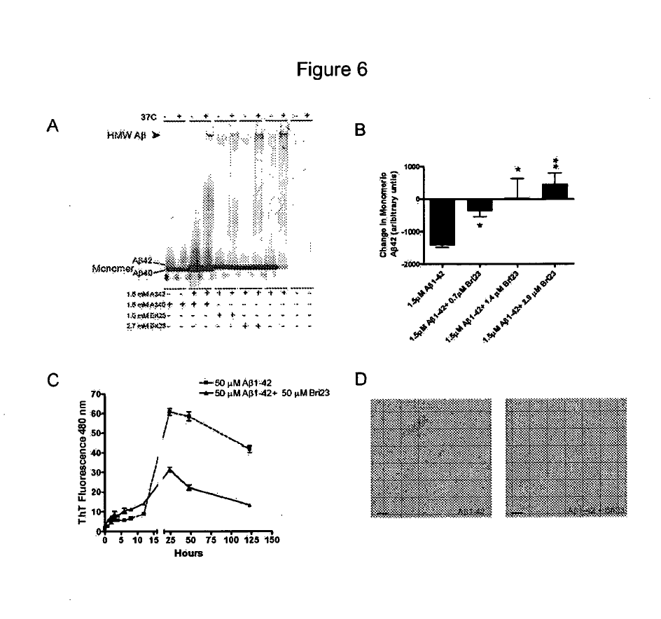

When Aβ1-42 aggregation was assessed using a native gel assay, Bri23 polypeptide inhibited Aβ42 aggregation. (FIGS. 6, A and, B). This inhibition was seen by both a modest reduction in high-molecular weight (HMW) Aβ1-42 aggregates and an increase in remaining monomeric Aβ1-42 (FIG. 6A), the later of which can be readily quantified (FIG. 6B). Notably, the effect is quite similar to that observed when 1.5 μM Aβ1-40 was incubated with 1.5 μM Aβ1-42. To further analyze the effect of Bri23 polypeptide on Aβ aggregation, the effect of equimolar concentrations (25 μM or 50 μM) of the Bri23 polypeptide on monomeric Aβ1-42 aggregation into Aβ1-42 fibrils or protofibrils using Thioflavin T (ThT) fluorescence was examined. As described elsewhere, prolonged incubations of Bri23 polypeptide, by itself, did not result in aggregation or β-sheet formation as assessed by ThT fluorescence, change in CD spectra or insolubility (Gibson et al., Biochem. Soc. Trans., 33: 1111 (2005)). Co-aggregation of Aβ42 and Bri23 demonstrates that Bri23 polypeptide appears to initially increase the rate of aggregate formation during the first 12 hours of incubation, but inhibits fibril formation at later time points (FIG. 6C). On average, after 120-200 hours of incubation, Bri23 polypeptide inhibited Aβ1-42 aggregation by an average of 46%±9% (n=6, p=0.0004). Atomic force microscopy (AFM) imaging confirmed the inhibitory effects of Bri23 polypeptide on Aβ1-42 aggregation in these assays (FIG. 6D). These results demonstrate that Bri23 polypeptide has a complex effect on aggregation of monomeric Aβ1-42; however, both assays are consistent with a net inhibitory effect of Bri23 polypeptide on amyloid formation presumably through inhibition of a later stage in fibril assembly.

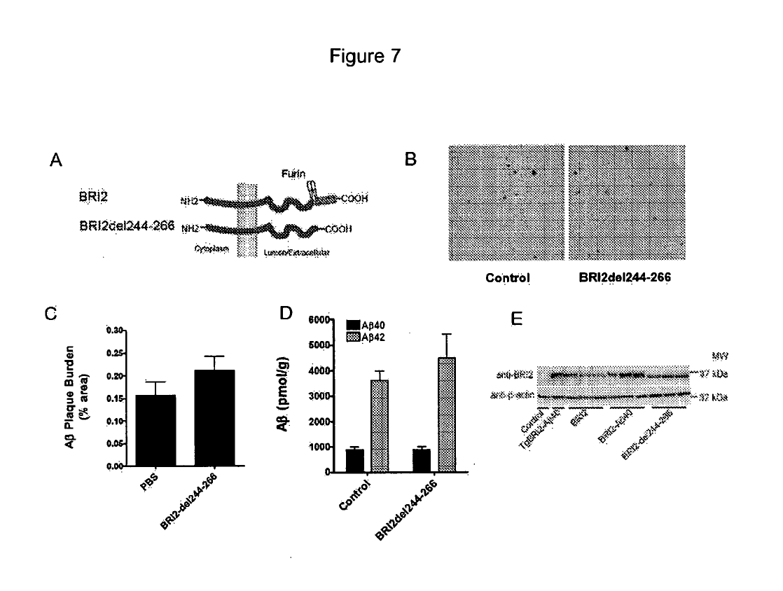

These results suggest that the anti-amyloidogenic effect of the BRI2 polypeptide is likely Bri23-Aβ interaction. To further test this, a cDNA that expresses a truncated BRI2 polypeptide lacking the Bri23 polypeptide sequence was generated (BRI2del244-266, FIG. 7A), and rAAV gene transfer was used to deliver this construct to P0 TgCRND8 mice. Transgene positive mice were killed at 3 months of age and biochemical and histochemical Aβ loads were examined. Analyses of Aβ loads revealed no significant difference between BRI2del244-266 and the control groups (FIG. 7B-D).

Western blot analyses of brain lysates demonstrated that the viral delivery method produced roughly equivalent expression levels from the BRI2 and BRI2del244-266 constructs and somewhat higher levels from BRI2Aβ1-40 (FIG. 7E). These later data and the lack of anti-amyloidogenic effect from BRI2del244-266 demonstrate that the Bri23 polypeptide sequence is critical for the inhibitory effect of BRI2 in vivo. Together with the data demonstrating that Bri23 polypeptide directly inhibits Aβ aggregation in vitro, these data support an anti-amyloidogenic, chaperone-like, function for the Bri23 polypeptide. The tested Bri23 polypeptide contained the sequence FENKF that is homologous to peptide-based Aβ aggregation inhibitors incorporating a FxxxF motif (Sato et al., Biochemistry, 45: 5503 (2006)). Moreover, solid state NMR analysis demonstrated direct binding of an eight amino acid sequence containing the FEGKF sequence with the G₃₃xxxG₃₇ segment of Aβ1-40, a sequence proposed to be critical for formation and stability of β-sheet structure (Sato et al., Biochemistry, 45:5503 (2006) and Liu et al., Biochemistry, 44: 3591 (2005)).

### Statistical Analysis

One-way analysis of variance (ANOVA) with post hoc Holm-Sidak multiple comparison test or two-tailed Student's t-test was used for statistical comparison (SigmaStat 3.0 version). If the data set did not meet the parametric test assumptions, non-parametric statistics was performed, either Kruskal-Wallis Test (One Way Analysis of Variance on Ranks) followed by post hoc Dunn's multiple comparison procedures or Mann-Whitney Rank Sum Test (SigmaStat 3.0 version). Variance was reported as standard error of the mean (s.c.m).

### Genetic Association Analysis

To further evaluate the pathophysiologic significance of these findings, six SNPs in the ITM2B gene that encodes BRI2 were analyzed for association with late onset AD (LOAD).

As demonstrated by the results presented in Table 1, all variants were checked and exhibited no evidence for departure from Hardy Weinberg equilibrium as indicated by the p value tabulated in column “H-W-P”. Single variants were analyzed by logistic regression with gender, age at diagnosis/entry, and ApoE ε4 (+/−) as covariates using subjects of all ages in the exploratory (JS), follow up (RS+AUT), and combined series. For each variant, dominant (12+22 vs. 11), recessive (22 vs. 12+22) and allelic dosage (11=0, 12=1, 22=2) models were assessed; p values are given for the model (“Best model” column) that was most significant. In the exploratory series no variant was significant at p=0.05 in any model. In the combined series, variants 984 (p=0.048, dominant model) and 1009 (p=0.034, recessive model) were significant at the 0.05 level. Variants 985 (p=0.063, recessive model), 986 (p=0.119, dominant model), and 987 (p=0.254, recessive model) also exhibited suggestive association. Thus, in the combined series, five of the six variants tested exhibited significant or suggestive association with modest ORs ranging from 0.73 to 1.48. The position of each variant is indicated relative to Human Genome Build 36.1. The major allele is shown in column “1”, the minor allele in column “2”, the minor allele frequency in column “MAF”. To evaluate whether each SNP is located in a region showing conservation between the mouse and human genomes, a sliding 100 by window encompassing the SNP was employed. The maximal % of bases that were identical (mouse vs. human) in a 100 by window encompassing the SNP is reported in the column labeled “Cons.”

The results for subjects of all ages and subjects with age at diagnosis/entry between 60-80 years are presented separately in Table 2 for the exploratory (JS), follow-up (RS-AUT), and combined series. Haplo Stats was employed to identify common haplotypes (frequency >1%). The allelic composition of each haplotype is presented in the “Haplotype” column, where 0 and 1 indicate the presence of a major or minor allele respectively for each of the 6 variants along the haplotype in the 5′→3′ orientation from the p to q telomere. The global p values presented for each series were determined using the score statistic implemented in Haplo Stats using gender, age at diagnosis/entry and ApoE ε4 (+/−) as covariates. Univariable logistic regression using the same covariates was employed to determine the OR, 95% confidence interval, and p value for each haplotype as compared to all others. Haplotypes are sorted ascending by OR in the combined series with ages of 60-80 years.

TABLE 1
Association of single ITM2B variants with LOAD.

| | | Chr | Gene | | | | | AD | | | Control | | | |
|---|---|---|---|---|---|---|---|---|---|---|---|---|---|---|
| ID | rs | Position | Location | Cons. | MAF | 1 | 2 | 11 | 12 | 22 | 11 | 12 | 22 | H-W-P |
| 984 | rs9332248 | 47704774 | 5′ Flank | 72% | 2% | C | G | 1739 | 68 | 1 | 1923 | 51 | 0 | 0.56 |
| 985 | rs1925744 | 47737742 | 3′ Flank | 65% | 36% | T | A | 1011 | 667 | 134 | 1110 | 751 | 120 | 0.42 |
| 986 | rs9332295 | 47731251 | Intron 5 | 61% | 3% | G | A | 1760 | 50 | 1 | 1902 | 65 | 2 | 0.07 |
| 987 | rs3803188 | 47706133 | Intron 1 | 87% | 26% | C | T | 1004 | 646 | 131 | 1070 | 757 | 128 | 0.30 |
| 1008 | rs9535001 | 47736640 | 3′ Flank | 71% | 2% | T | G | 1649 | 150 | 5 | 1787 | 177 | 5 | 0.45 |
| 1009 | rs9534996 | 47698950 | 5′ Flank | 12% | 28% | G | A | 926 | 716 | 156 | 1024 | 786 | 141 | 0.74 |

| | | Exploratory | | | Follow-up | | | | | |
|---|---|---|---|---|---|---|---|---|---|---|
| | Best | Series (JS) | | | Series (RS + AUT) | | | Combined Series | | |
| ID | model | OR | 95% CI | P | OR | 95% CI | P | OR | 95% CI | P |
| 984 | dominant | 0.71 | 0.38 to 1.35 | 0.298 | 2.28 | 1.36 to 3.83 | 0.002 | 1.48 | 1.00 to 2.19 | 0.048 |
| 985 | recessive | 1.44 | 0.89 to 2.35 | 0.144 | 1.24 | 0.89 to 1.72 | 0.210 | 1.30 | 0.99 to 1.70 | 0.063 |
| 986 | dominant | 0.96 | 0.47 to 1.96 | 0.911 | 0.64 | 0.39 to 1.04 | 0.074 | 0.73 | 0.49 to 1.08 | 0.119 |
| 987 | recessive | 1.54 | 0.97 to 2.45 | 0.070 | 1.01 | 0.72 to 1.41 | 0.964 | 1.17 | 0.89 to 1.53 | 0.254 |
| 1008 | dominant | 0.89 | 0.57 to 1.38 | 0.599 | 1.03 | 0.77 to 1.37 | 0.851 | 0.98 | 0.77 to 1.24 | 0.847 |
| 1009 | recessive | 1.49 | 0.95 to 2.35 | 0.086 | 1.24 | 0.91 to 1.69 | 0.167 | 1.32 | 1.02 to 1.69 | 0.034 |

TABLE 2
Association of ITM2B haplotypes with LOAD.

| | | All ages | | | | | | | | | | | |
|---|---|---|---|---|---|---|---|---|---|---|---|---|---|
| | | Exploratory | | | | Follow-up | | | | | | | |
| | | Series (JS) | | | | Series (RS + AUT) | | | | Combined Series | | | |
| | | Global p = 0.13 | | | | Global p = 0.003 | | | | Global p = 0.045 | | | |
| ID | Haplotype | Freq | OR | 95% CI | P | Freq | OR | 95% CI | P | Freq | OR | 95% CI | P |
| H8 | H101000 | 0.01 | 1.56 | 0.64 to 3.79 | 0.33 | 0.01 | 0.32 | 0.14 to 0.73 | 0.01 | 0.01 | 0.60 | 0.34 to 1.06 | 0.08 |
| H4 | H000100 | 0.02 | 1.04 | 0.49 to 2.20 | 0.92 | 0.02 | 0.70 | 0.43 to 1.13 | 0.14 | 0.02 | 0.79 | 0.53 to 1.18 | 0.25 |
| H1 | H000000 | 0.64 | 0.89 | 0.73 to 1.07 | 0.20 | 0.65 | 0.99 | 0.88 to 1.13 | 0.91 | 0.65 | 0.96 | 0.86 to 1.06 | 0.42 |
| H3 | H000010 | 0.04 | 0.93 | 0.61 to 1.43 | 0.75 | 0.05 | 1.17 | 0.88 to 1.56 | 0.28 | 0.05 | 1.07 | 0.85 to 1.36 | 0.56 |
| H7 | H001001 | 0.01 | 1.46 | 0.66 to 3.24 | 0.35 | 0.01 | 1.00 | 0.50 to 1.99 | 1.00 | 0.01 | 1.13 | 0.68 to 1.89 | 0.63 |
| H2 | H101001 | 0.24 | 1.06 | 0.86 to 1.31 | 0.56 | 0.24 | 0.96 | 0.83 to 1.10 | 0.52 | 0.24 | 0.99 | 0.88 to 1.12 | 0.90 |
| H5 | H110000 | 0.02 | 0.78 | 0.42 to 1.47 | 0.45 | 0.01 | 2.34 | 1.37 to 4.00 | 0.002 | 0.02 | 1.53 | 1.03 to 2.28 | 0.04 |
| H6 | H100000 | 0.01 | 4.49 | 1.49 to 13.53 | 0.01 | 0.01 | 1.21 | 0.72 to 2.04 | 0.47 | 0.01 | 1.61 | 1.02 to 2.52 | 0.04 |

| | 60-80 | | | | | | | | | | | |
|---|---|---|---|---|---|---|---|---|---|---|---|---|
| | Exploratory | | | | Follow-up | | | | | | | |
| | Series (JS) | | | | Series (RS + AUT) | | | | Combined Series | | | |
| | Global p = 0.272 | | | | Global p = 0.002 | | | | Global p = 0.006 | | | |
| ID | Freq | OR | 95% CI | P | Freq | OR | 95% CI | P | Freq | OR | 95% CI | P |
| H8 | 0.01 | 0.82 | 0.23 to 2.94 | 0.76 | 0.01 | 0.35 | 0.13 to 0.99 | 0.05 | 0.01 | 0.49 | 0.23 to 1.08 | 0.08 |
| H4 | 0.02 | 1.27 | 0.52 to 3.06 | 0.60 | 0.02 | 0.50 | 0.26 to 0.96 | 0.04 | 0.02 | 0.67 | 0.40 to 1.10 | 0.11 |
| H1 | 0.63 | 0.85 | 0.66 to 1.10 | 0.22 | 0.65 | 0.90 | 0.76 to 1.07 | 0.24 | 0.65 | 0.89 | 0.78 to 1.03 | 0.11 |
| H3 | 0.05 | 0.74 | 0.43 to 1.26 | 0.26 | 0.05 | 1.14 | 0.78 to 1.67 | 0.49 | 0.05 | 0.98 | 0.72 to 1.32 | 0.87 |
| H7 | 0.02 | 1.65 | 0.62 to 4.38 | 0.31 | 0.01 | 0.87 | 0.34 to 2.24 | 0.77 | 0.01 | 1.05 | 0.55 to 2.01 | 0.87 |
| H2 | 0.25 | 1.13 | 0.85 to 1.49 | 0.40 | 0.24 | 1.08 | 0.89 to 1.30 | 0.45 | 0.24 | 1.10 | 0.94 to 1.29 | 0.22 |
| H5 | 0.01 | 1.11 | 0.43 to 2.90 | 0.83 | 0.01 | 3.99 | 1.79 to 8.87 | 0.001 | 0.01 | 2.27 | 1.26 to 4.08 | 0.01 |
| H6 | 0.01 | 5.02 | 1.15 to 21.86 | 0.03 | 0.01 | 1.86 | 0.79 to 4.35 | 0.15 | 0.01 | 2.35 | 1.18 to 4.68 | 0.02 |

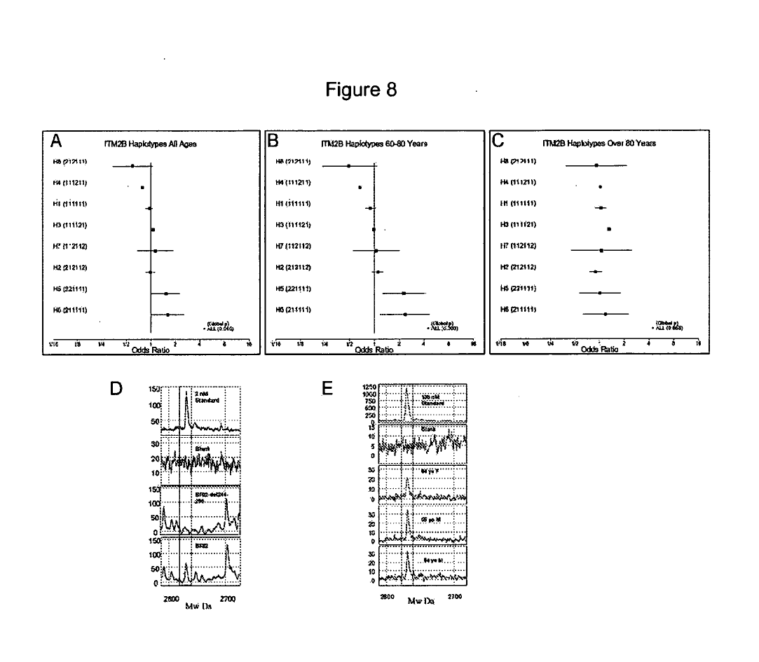

The eight ITM2B haplotypes shown in Table 2 and FIG. 8 pair to form 36 genotypes. Many of these genotypes are extremely rare because five of the ITM2B haplotypes have frequencies of 2% or less (Table 2). In 60-80 year old subjects in the combined series, there were 14 multilocus genotypes that occurred 10 times or more. The genotype of the single variants comprising each multilocus genotype are presented in the “MLG” column of Table 3, where 0 (major allele homozygote), 1 (heterozygote), and 2 (minor allele homozygote) indicate the number of minor alleles respectively in the genotypes for each of the 6 variants arranged in the 5′→3′ orientation from the p to q telomere. Analysis of these genotypes by Haplo Stats, which employs an expectation maximization algorithm, revealed that each of the 14 MLGS was formed by one haplotype pair with a probability over 99%. The haplotype pair forming each MLG is presented in the “Haplo pair” column of Table 3. Using logistic regression with gender, age at diagnosis/entry, and ApoE ε4 (+/−) as covariates, global (multivariable regression), and individual (univariable regression) p values were determined for the 14 MLGs, which accounted for 97.7% of all subjects. The rare MLGs, which accounted for the remaining 2.3% of subjects, were pooled and included in the analysis as an additional group. The results for these 15 MLG groups, which had a global p=0.052 in the combined series, were tabulated; the ORs, 95% CIs, and p values for MLGs in the combined series were obtained by univariable logistic regression comparing subjects with each MLG to all others. In the exploratory (JS) and follow-up (RS-AUT) series, ORs, 95% CIs and p values were determined in the same way for MLGs occurring at least 10 times. In the combined series, the H1/H5 (p=0.039), H2/H2 (p=0.050), H1/H6 (p=0.051), and H2/H5 (p=0.14) genotypes, which form a high risk group with significant or suggestive association, had ORs of 2.1, 1.5, 2.7, and 3.0 respectively. The H1/H4 (p=0.023) and H1/H8 (p=0.14) genotypes, which form a low risk group, had significant or suggestive ORs of 0.50 and 0.53 respectively. The remaining nine genotypes, which form an intermediate group, had ORs (0.91<OR<1.27) that were not significant (0.35,p,0.97).

TABLE 3
Association of ITM2B multilocus genotypes with LOAD.

| | | | 60-80 | | | | | | | | | | |
|---|---|---|---|---|---|---|---|---|---|---|---|---|---|
| | | | Exploratory Series | | | | | | | Validation Series | | | |
| | | Haplo | | | n. | n. | | | | | | n. | n. |
| ID | MLG | Pair | Freq | n | ad | con | OR | 95% CI | p | Freq | n | ad | con |
| 1 | MLG000100 | H1/H4 | 0.03 | 20 | 13 | 7 | 1.20 | 0.47 to 3.40 | 0.648 | 0.02 | 35 | 9 | 26 |
| 2 | MLG101000 | H1/H8 | 0.02 | 11 | 4 | 7 | 0.61 | 0.15 to 2.40 | 0.475 | 0.01 | 17 | 6 | 11 |
| 6 | MLG000000 | H1/H1 | 0.39 | 263 | 124 | 139 | 0.84 | 0.59 to 1.19 | 0.317 | 0.43 | 616 | 304 | 312 |
| 8 | MLG000020 | H3/H3 | 0.01 | 5 | 1 | 4 | N/A | N/A | N/A | 0.00 | 3 | 2 | 1 |
| 5 | MLG202001 | H2/H8 | 0.00 | 1 | 1 | 0 | N/A | N/A | N/A | 0.00 | 3 | 0 | 3 |
| 3 | MLG101001 | H1/H2 | 0.32 | 216 | 115 | 101 | 0.93 | 0.65 to 1.34 | 0.711 | 0.31 | 447 | 210 | 237 |
| 4 | MLG101011 | H2/H3 | 0.03 | 17 | 8 | 9 | 1.10 | 0.37 to 3.27 | 0.860 | 0.02 | 35 | 15 | 20 |
| 10 | MLG101101 | H2/H4 | 0.00 | 3 | 1 | 2 | N/A | N/A | N/A | 0.01 | 7 | 2 | 5 |
| 13 | MLG000010 | H1/H3 | 0.05 | 34 | 16 | 18 | 0.98 | 0.46 to 2.11 | 0.965 | 0.06 | 85 | 41 | 44 |
| 9 | MLG102002 | H2/H7 | 0.01 | 8 | 4 | 4 | N/A | N/A | N/A | 0.01 | 9 | 6 | 3 |
| 7 | MLG201001 | H2/H6 | 0.00 | 3 | 3 | 0 | N/A | N/A | N/A | 0.01 | 11 | 4 | 7 |
| 12 | MLG001001 | H1/H7 | 0.02 | 12 | 8 | 4 | 2.51 | 0.68 to 9.25 | 0.165 | 0.01 | 11 | 5 | 6 |
| 14 | MLG100001 | H1/H9 | 0.00 | 3 | 1 | 2 | N/A | N/A | N/A | 0.00 | 4 | 3 | 1 |
| 18 | MLG.rare | Rare | 0.01 | 7 | 5 | 2 | N/A | N/A | N/A | 0.01 | 12 | 7 | 5 |
| 11 | MLG211001 | H2/H5 | 0.01 | 4 | 1 | 3 | N/A | N/A | N/A | 0.00 | 6 | 6 | 0 |
| 18 | MLG100000 | H1/H6 | 0.01 | 9 | 1 | 2 | N/A | N/A | N/A | 0.01 | 17 | 13 | 4 |
| 16 | MLG202002 | H2/H2 | 0.05 | 39 | 22 | 1 | 1.43 | 0.72 to 3.05 | 0.267 | 0.00 | 62 | 47 | 35 |
| 17 | MLG110000 | H1/H6 | 0.02 | 12 | 1 | 1 | 1.49 | 0.37 to 4.86 | 0.649 | 0.02 | 32 | 25 | 9 |

| | 60-80 | | | | | | | | | |
|---|---|---|---|---|---|---|---|---|---|---|
| | | | | Combined Series | | | | | | |
| | Validation Series | | | | | n. | n. | | | |
| ID | OR | 95% CI | p | Freq | n | ad | con | OR | 95% CI | p |
| 1 | 0.29 | 0.13 to 0.07 | 0.003 | 0.03 | 55 | 22 | 33 | 0.50 | 0.26 to 0.97 | 0.023 |
| 2 | 0.48 | 0.16 to 1.42 | 0.182 | 0.01 | 28 | 10 | 18 | 0.53 | 0.23 to 1.24 | 0.145 |
| 6 | 0.96 | 0.76 to 1.20 | 0.694 | 0.42 | 879 | 428 | 451 | 0.91 | 0.76 to 1.10 | 0.350 |
| 8 | N/A | N/A | N/A | 0.00 | 8 | 3 | 5 | 0.54 | 0.12 to 2.54 | 0.438 |
| 5 | N/A | N/A | N/A | 0.00 | 4 | 1 | 3 | 0.44 | 0.04 to 5.02 | 0.510 |
| 3 | 0.90 | 0.70 to 1.15 | 0.410 | 0.31 | 663 | 325 | 338 | 0.94 | 0.77 to 1.15 | 0.537 |
| 4 | 0.92 | 0.44 to 1.91 | 0.814 | 0.03 | 52 | 23 | 29 | 0.94 | 0.52 to 1.71 | 0.839 |
| 10 | N/A | N/A | N/A | 0.01 | 10 | 3 | 7 | 0.97 | 0.25 to 3.79 | 0.967 |
| 13 | 1.08 | 0.67 to 1.75 | 0.752 | 0.06 | 119 | 57 | 62 | 1.03 | 0.69 to 1.54 | 0.880 |
| 9 | N/A | N/A | N/A | 0.01 | 17 | 10 | 7 | 1.27 | 0.44 to 3.65 | 0.661 |
| 7 | 0.76 | 0.19 to 3.08 | 0.698 | 0.01 | 14 | 7 | 7 | 1.30 | 0.42 to 4.07 | 0.653 |
| 12 | 0.61 | 0.16 to 2.28 | 0.457 | 0.01 | 23 | 13 | 10 | 1.26 | 0.51 to 3.11 | 0.624 |
| 14 | N/A | N/A | N/A | 0.00 | 7 | 4 | 3 | 1.57 | 0.31 to 7.92 | 0.583 |
| 18 | 1.32 | 0.37 to 4.68 | 0.671 | 0.01 | 28 | 16 | 12 | 1.35 | 0.60 to 3.07 | 0.469 |
| 11 | N/A | N/A | N/A | 0.01 | 10 | 7 | 3 | 2.97 | 0.70 to 12.58 | 0.140 |
| 18 | 9.07 | 0.90 to 10.49 | 0.074 | 0.01 | 26 | 20 | 6 | 2.60 | 0.99 to 7.11 | 0.051 |
| 16 | 1.40 | 0.91 to 2.44 | 0.110 | 1.00 | 121 | 69 | 52 | 1.40 | 1.00 to 2.23 | 0.050 |
| 17 | 2.52 | 1.33 to 5.26 | 0.026 | 0.02 | 14 | 30 | 14 | 2.07 | 1.04 to 4.13 | 0.039 |

### Genotyping

Genotyping was performed on an ABI 7900 instrument using TaqMan chemistry with primers and probes designed by Applied Biosystems.

Analysis of ITM2B mRNA

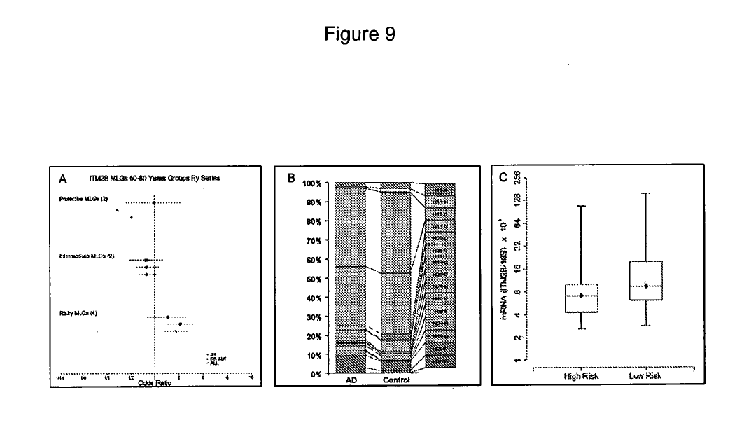

Total RNA was extracted from samples of cerebellum from 141 AD brains using an ABI PRISM 6100 Nucleic Acid PrepStation and the Total RNA Isolation Chemistry kit from Applied Biosystems. RNA was reverse transcribed (RT) to single-stranded cDNA using the High-Capacity cDNA Archive Kit from Applied Biosystems. Rcal-Time Quantitative PCR was performed in triplicate for each sample using ABI TaqMan Low Density expression Arrays (384-Well Micro Fluidic Cards) with a pre-validated TaqMan Gene Expression Assay. 18s RNA was used as the endogenous control for the relative quantitation of ITM2B mRNA. Data analysis was performed using ABI PRISM® SDS software 2.2 version. Average delta Ct values were used to express IDE mRNA levels as IDE/18s×10-4 (FIG. 9).

These six ITM2B SNPs, which are in strong linkage disequilibrium, formed eight haplotypes that accounted for more that 99% of all ITM2B genes in the American Caucasians that were examined. These eight haplotypes were analyzed in large exploratory (563 AD, 563 Control) and follow-up (1130 AD, 1328 Control) case-control series. None of the haplotypes had odds ratios (ORs) that were significantly (p<0.05) different in the two series (Table 2). In the combined series, the global p value for haplotypic association, determined with the score statistic implemented in Haplo Stats using gender, age at onset/entry and ApoE (+/−) as covariates, was significant (p=0.042) (FIG. 8A). The effect of both risky and protective haplotypes was stronger in subjects with an age at diagnosis/entry of 80 years or less (FIG. 8B), an age dependence similar to that for the well established ApoE haplotypes (Farrer et al., JAMA, 278: 1349 (1997)). In subjects over 80 (643 AD, 842 Control), where haplotypes had weak effects (FIG. 8C), association with LOAD no longer achieved significance (global p=0.66). In the 60-80 year group (1050 AD, 1059 Control) where effects were strong, the significance of haplotypic association improved considerably (global p=0.006) compared to subjects of all ages (1693 AD, 1891 Control) despite the reduced number of subjects (FIG. 8B). The haplotypes in the exploratory and follow-up series showed good replication in the 60-80 year group (Table 2), there was no significant (p<0.05) evidence that the odds ratio (OR) for any haplotype was different between the two series. Although suggestive, haplotypic association did not achieve significance in the smaller exploratory series (global p=0.27), but it was significant in the large follow up series (global p=0.002). Analysis of the 60-80 year group in the combined series (Table 2, FIG. 8B) revealed that H5 (p=0.006), and H6 (p=0.020) were significantly risky with ORs of 2.3 and 2.4 respectively. H2 (p=0.22) showed suggestive association with a risky OR of 1.1. H1 (p=0.11), H4 (p=0.11), and H8 (p=0.08) exhibited suggestive association with protective ORs of 0.89, 0.67 and 0.49 respectively. Thus, in the combined series, 6 of the 8 ITM2B haplotypes showed significant or suggestive association in the 60-80 year group (Table 2, FIG. 8B).

The two ITM2B haplotypes inherited by each individual form a genotype which interacts with other genetic factors and the environment to determine the effect of the ITM2B gene on that individual's risk for LOAD. The association of ITM2B genotypes with LOAD is analyzed and discussed in the online supplementary material (Table 3). In the combined series, the H1/H5 (p=0.039), H2/H2 (p=0.05), H1/H6 (p=0.051), and H2/H5 (p=0.14) genotypes exhibited significant or suggestive association with risky ORs of 2.1, 1.5, 2.7, and 3.0 respectively. This set of 4 risky MLGs was found in 12.0% of AD and 7.1% of control subjects (FIG. 3, panel B) and had a combined OR of 1.83 (1.32-2.53) when analyzed by logistic regression using gender, age at onset/entry and ApoE (+/−) as covariates. To determine if this set of genotypes is associated with altered expression of the ITM2B gene, ITM2B mRNA levels in the cerebellum, a region with minimal AD pathology where mRNA levels are not altered by the profound neuronal loss and gliosis found in affected brain regions at autopsy, were analyzed. Using real time PCR with 18s RNA as reference, ITM2B mRNA was analyzed in the cerebellum of 141 AD brains. ITM2B mRNA levels were significantly increased by 32% in the 116 subjects with low risk genotypes as compared to the 25 subjects with high risk genotypes (p=0.02 by two sided Mann Whitney test).

### Statistical Analysis of Genetic Association

The genotypes of all six variants were checked and revealed no significant evidence for departure from Hardy Weinberg equilibrium. Single variants were analyzed by logistic regression with gender, age at diagnosis/entry, and ApoE ε4 (+/−) as covariates. For each variant, dominant (12+22 vs. 11), recessive (22 vs. 12+22) and allelic dosage (11=0, 12=1, 22=2) models were assessed. Haplo Stats was employed to identify common haplotypes (frequency >1%). Global p values for haplotypic association were determined using the score statistic implemented in Haplo Stats using gender, age at diagnosis/entry and ApoE ε4 (+/−) as covariates. Univariable logistic regression using the same covariates was employed to determine the odds ratio, 95% confidence interval, and p value for each haplotype as compared to all others. In 60-80 year old subjects in the combined series, the six ITM2B variants formed 14 multilocus genotypes that occurred 10 times or more. Analysis of these genotypes by Haplo Stats, which employs an expectation maximization algorithm, revealed that each of the 14 MLGs was formed by one haplotype pair with a probability over 99%. The rare MLGs, which accounted for the remaining 2.3% of subjects, were pooled and included in the analysis as an additional group. A global p value for association of these 15 MLG groups with LOAD was obtained by multivariable logistic regression with gender, age at diagnosis/entry, and ApoE ε4 (+/−) as covariates. Univariable logistic regression using the same covariates was employed to determine the odds ratio, 95% confidence interval, and p value for each MLG (haplotype pair) as compared to all others.

Collectively, these results indicate that ITM2B has multiple variants that influence ITM2B mRNA levels and risk for LOAD.

A high performance liquid chromatography/HPLC Mass spectrometry (HPLC/MS)-based method to detect secreted Bri23 polypeptide was developed. Using this approach, Bri23 polypeptide secretion from cells transfected with BRI2 but not BRIdel244-266 was detected (FIG. 8D). More, significantly, Bri23 polypeptide in human CSF was detected, indicating that Bri23 polypeptide is produced in vivo (FIG. 8E).

Collectively, the results provided herein demonstrate the existence of a pathologically relevant, extracellular polypeptide quality control mechanism mediated by the production and secretion of an anti-amyloidogenic Bri23 polypeptide derived from a BRI2 polypeptide. In FDD brains, Aβ and the ADan polypeptides are co-deposited and bind to each other in vitro (Akiyama et al., Acta. Neuropathol. (Berl), 107:53 (2004)). These findings suggest that the FDD linked BRI2 mutation may corrupt a normally protective anti-amyloidogenic mechanism resulting in co-aggregation of the mutant polypeptide with a normal binding partner.

The robust inhibitory effect of BRI2 on Aβ aggregation both in vitro and in vivo and the association of its genetic variants with mRNA levels and AD indicate that BRI2 is a factor that influences risk for AD by modulating Aβ aggregation and deposition. These results also support an approach to AD therapy or prevention based on increasing levels of the Bri23 polypeptide in brain.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for reducing Aβ aggregation in a mammal, wherein said method comprises administering a composition, to said mammal, under conditions wherein Aβ aggregation in said mammal is reduced, wherein said composition comprises a BRI polypeptide or a fragment of a BRI polypeptide.

2. The method of claim 1, wherein said composition comprises a Bri23 polypeptide, a Bri24 polypeptide, or a Bri25 polypeptide.

3. The method of claim 2, wherein said Bri23, Bri24, or Bri25 polypeptide comprises a D-amino acid.

4. The method of claim 2, wherein each amino acid of said Bri23, Bri24, or Bri25 polypeptide is a D-amino acid.

5. The method of claim 1, wherein said BRI polypeptide or said fragment is unmodified.

6. The method of claim 1, wherein said BRI polypeptide or said fragment is reduced.

7. The method of claim 1, wherein said BRI polypeptide or said fragment contains an intrachain disulfide bond.

8. The method of claim 1, wherein said BRI polypeptide or said fragment comprises one or more unnatural or modified amino acids that increase brain levels.

9. The method of claim 1, wherein said composition comprises a Bri23 polypeptide having an intrachain disulfide bond between Cys5 and Cys22.

10. The method of claim 1, wherein said composition comprises a Bri24 polypeptide having an intrachain disulfide bond between Cys5 and Cys22.

11. The method of claim 1, wherein said composition comprises a Bri25 polypeptide having an intrachain disulfide bond between Cys5 and Cys22.

12. A method for reducing Aβ aggregation in a mammal, wherein said method comprises administering a composition, to said mammal, under conditions wherein Aβ aggregation in said mammal is reduced, wherein said composition comprises a BRI2 polypeptide, a fragment of said BRI2 polypeptide, a nucleic acid encoding said BRI2 polypeptide, or a nucleic acid encoding said fragment.

13. The method of claim 12, wherein said mammal is a human.

14. The method of claim 12, wherein said mammal has Alzheimer's disease.

15. The method of claim 12, wherein said composition comprises said BRI2 polypeptide.

16. The method of claim 12, wherein said composition comprises said fragment.

17. The method of claim 16, wherein said fragment is a Bri23 polypeptide.

18. The method of claim 12, wherein said composition comprises nucleic acid encoding said BRI2 polypeptide.

19. The method of claim 12, wherein said composition comprises nucleic acid encoding said fragment.

20. The method of claim 19, wherein said fragment is a Bri23 polypeptide.

21. A method for reducing Aβ aggregation in a mammal, wherein said method comprises administering a composition, to said mammal, under conditions wherein Aβ aggregation in said mammal is reduced, wherein said composition comprises an agent that increases expression of a BRI polypeptide in said mammal.

22. The method of claim 21, wherein said composition comprises the ability to increase expression of a fragment of a BRI polypeptide, wherein said fragment comprises at least 15 amino acid residues from the carboxyl terminus of a full length BRI polypeptide.

23. A method for reducing Aβ aggregation in a mammal, wherein said method comprises administering a composition, to said mammal, under conditions wherein Aβ aggregation in said mammal is reduced, wherein said composition comprises an agent that increases proteolytic cleavage of a BRI polypeptides to increase the levels of a fragment of said BRI polypeptide in said mammal.

24. The method of claim 23, wherein said agent is a nucleic acid encoding a protease.

25. The method of claim 24, wherein said protease is a furin protease.


SEQUENCE LISTING (9 sequences)

SEQ ID NO: 1 - protein, 263 aa, Homo sapiens [sequence omitted]

SEQ ID NO: 2 - protein, 160 aa, Homo sapiens
MVKVTFNSALAQKEAKKDEPKSGEEALIIPPDAVAVDCKDPDDVVPVGQRRAWCWCMCFGLAFMLAGVILGGAYLYKYFALQAGTYLPQSYLIHEHMVITDRIENIDHLGFFIYRLCHDKETYKLQRRETIKGIQKREASNCFAIRHFENKFAVETLICS

SEQ ID NO: 3 - protein, 267 aa, Homo sapiens [sequence omitted]

SEQ ID NO: 4 - protein, 23 aa, Homo sapiens
EASNCFAIRHFENKFAVETLICS

SEQ ID NO: 5 - protein, 24 aa, Homo sapiens
AIDKCWKIRHFPNEFIVETKICQD

SEQ ID NO: 6 - protein, 25 aa, Homo sapiens
GAKNCNAIRHFENTFVVETLICGVV

SEQ ID NO: 7 - protein, 137 aa, Homo sapiens
MVKVTFNSALAQKEAKKDEPKSGEEALIIPPDAVAVDCKDPDDVVPVGQRRAWCWCMCFGLAFMLAGVILGGAYLYKYFALQAGTYLPQSYLIHEHMVITDRIENIDHLGFFIYRLCHDKETYKLQRRETIKGIQKR

SEQ ID NO: 8 - protein, 128 aa, Homo sapiens
MVKVTFNSALAQKEAKKDEPKSGEEALIIPPDAVAVDCKDPDDVVPVGQRRAWCWCMCFGLAFMLAGVILGGAYLYKYFALQAGTYLPQSYLIHEHMVITDRIENIDHLGFFIYRLCHDKETYKLQRR

SEQ ID NO: 9 - protein, 109 aa, Homo sapiens
MVKVTFNSALAQKEAKKDEPKSGEEALIIPPDAVAVDCKDPDDVVPVGQRRAWCWCMCFGLAFMLAGVILGGAYLYKYFALQAGTYLPQSYLIHEHMVITDRIENIDHL